(19) United States
(12) Patent Application Publication
(10) Pub. No.: US 2017/0014031 A1
(43) Pub. Date: Jan. 19, 2017

(54) SCANNER FOR PHOTO-ACOUSTIC TOMOGRAPHY AND PHOTO-ACOUSTIC TOMOGRAPHY APPARATUS USING SAME

(71) Applicant: POSTECH ACADEMY-INDUSTRY FOUNDATION, Pohang-si (KR)

(72) Inventors: Geun Bae LIM, Pohang-si (KR); Man Sik JEON, Daegu (KR); Chang Ho LEE, Daegu (KR); Jin Young KIM, Busan (KR); Chul Hong KIM, Pohang-si (KR)

(21) Appl. No.: 15/124,511

(22) PCT Filed: Dec. 23, 2014
(86) PCT No.: PCT/KR2014/012742

§ 371 Date: Sep. 8, 2016

(30) Foreign Application Priority Data
Mar. 25, 2014 (KR) 10-2014-0034809

(51) Int. Cl. A61B 5/00; G01N 29/24; G01N 29/26; A61B 1/313

(52) U.S. Cl. CPC A61B 5/0095; A61B 1/3132; G01N 29/2418; G01N 29/26; G01N 2291/02475

(57) ABSTRACT

Provided is a scanner for photoacoustic tomography including: a mirror which reflects light and a photoacoustic signal for the photoacoustic tomography; a first driving member which is attached with the mirror; a second driving member which is connected to the first driving member; first driving force supply units which are disposed under two ends of the first driving member in a first direction to exert a driving force for allowing the first driving member to perform tilting movement in the first direction; and second driving force supply units which are disposed under two ends of the second driving member in a second direction to exert a driving force for allowing the second driving member to perform tilting movement in the second direction.

### BACKGROUND OF THE INVENTION

1. Field of the Invention

The present invention relates to a photoacoustic tomography apparatus and, more particularly, to a scanner for photoacoustic tomography capable of being manufactured in so a small size as to be used for an endoscope, a microscope, or a laparoscope for surgery and capable of scanning simultaneously with light and a photoacoustic signal at a high scanning speed and a photoacoustic tomography apparatus having the scanner.

2. Description of the Related Art

A photoacoustic tomography apparatus is a new image processing method combined with an optical system and an ultrasonic system.

In a photoacoustic tomography technique, when a biological tissue is irradiated with light, the biological tissue absorbs light energy. The biological tissue absorbing the light energy is thermoelastically expanded, and thus, a photoacoustic signal (photoacoustic wave) is generated. The photoacoustic tomography apparatus acquires the generated photoacoustic signal through an ultrasonic transducer and processes the acquired photoacoustic signal to generate tomography image information.

In order to acquire three-dimensional photoacoustic tomography image, an objective lens and an ultrasonic transducer scan an observation object in plane direction by using a 2-axis linear stage, or in a state where an observation object is fixed by a stage an objective lens and an ultrasonic transducer scan directly the observation object on the stage. Namely, a three-dimensional photoacoustic tomography image is acquired by sequentially obtaining and combining one-dimensional depth-directional photoacoustic signals moving in the plane direction.

The linear stage configured with a stepping motor has a large size of several to several tens of centimeters, and thus, there is a limitation in increasing the scanning range and the scanning speed. In addition, since a wide range needs to be scanned in order to acquire a wide range of the photoacoustic tomography image, there is a problem in that a long time is taken to acquire the three-dimensional photoacoustic tomography image.

Therefore, in the related art, there has been a demand for development of a technique capable of manufacturing the scanner in a small size so as to be used for an endoscope, a microscope or a laparoscope for surgery, easily extending the scanning range, and improving the scanning speed.

### SUMMARY OF THE INVENTION

The invention is to provide a scanner for photoacoustic tomography capable of being manufactured in so a small size as to be used for an endoscope, a microscope, or a laparoscope for surgery and capable of scanning simultaneously with light and a photoacoustic signal at a high scanning speed and a photoacoustic tomography apparatus having the scanner.

According to an aspect of the invention, there is provided a scanner for photoacoustic tomography including: a mirror which reflects light and a photoacoustic signal for the photoacoustic tomography; a first driving member which is attached with the mirror; a second driving member which is connected to the first driving member; first driving force supply units which are disposed under two ends of the first driving member in a first direction to exert a driving force for allowing the first driving member to perform tilting movement in the first direction; and second driving force supply units which are disposed under two ends of the second driving member in a second direction to exert a driving force for allowing the second driving member to perform tilting movement in the second direction.

According to another aspect of the invention, there is provided a scanner for photoacoustic tomography including: an ultrasonic transducer which receives a photoacoustic signal; a first driving member which is attached with the ultrasonic transducer; a second driving member which is connected to the first driving member; first driving force supply units which are disposed under two ends of the first driving member in a first direction to exert a driving force for allowing the first driving member to perform tilting movement in the first direction; and second driving force supply units which are disposed under two ends of the second driving member in a second direction to exert a driving force for allowing the second driving member to perform tilting movement in the second direction.

According to still another aspect of the invention, there is provided a scanner for photoacoustic tomography including: an optical fiber which receives light for photoacoustic tomography and emits the light; a first driving member which is attached with a light emitting surface of the optical fiber a second driving member which is connected to the first driving member; first driving force supply units which are disposed under two ends of the first driving member in a first direction to exert a driving force for allowing the first driving member to perform tilting movement in the first direction; and second driving force supply units which are disposed under two ends of the second driving member in a second direction to exert a driving force for allowing the second driving member to perform tilting movement in the second direction.

According to further still another aspect of the invention, there is provided a scanner for photoacoustic tomography including: an ultrasonic transducer which receives a photoacoustic signal and where a hole is formed on a photoacoustic signal receiving surface; a first driving member which is attached with the ultrasonic transducer; a second driving member which is connected to the first driving member; first driving force supply units which are disposed under two ends of the first driving member in a first direction to exert a driving force for allowing the first driving member to perform tilting movement in the first direction; and second driving force supply units which are disposed under two ends of the second driving member in a second direction to exert a driving force for allowing the second driving member to perform tilting movement in the second direction, wherein the light emitting surface of the optical fiber is inserted into the hole of the ultrasonic transducer to be coupled.

According to the invention, it is possible to obtain effects that a scanner for photoacoustic tomography can be manufactured in a small size to be used for an endoscope, a microscope, or a laparoscope for surgery, the scanner can be used for an endoscope or a laparoscope, a transmission path of light and a photoacoustic signal can be changed at a high speed, and a scanning range can be easily extended.

### BRIEF DESCRIPTION OF THE DRAWINGS

FIG. 1 is a schematic diagram illustrating a configuration of a photoacoustic tomography apparatus according to a first embodiment of the invention;

FIGS. 2A and 2B are diagrams illustrating a configuration of a scanner illustrated in FIG. 1;

FIGS. 3A and 3B are diagrams illustrating operations of the scanner illustrated in FIG. 1;

FIGS. 4A and 4B are diagrams illustrating an outer appearance of the scanner illustrated in FIG. 1;

FIGS. 5A and 5B are diagrams illustrating results of photoacoustic tomography according to the first embodiment of the invention;

FIG. 6 is a flowchart illustrating a process of driving a scanner of the photoacoustic tomography apparatus according to the first embodiment of the invention;

FIG. 7 is a schematic diagram illustrating a configuration of a photoacoustic tomography apparatus according to a second embodiment of the invention; and

FIGS. 8A and 8B are diagrams illustrating a configuration of the scanner illustrated in FIG. 7.

### DETAILED DESCRIPTION OF THE PREFERRED EMBODIMENTS

According to the invention, a scanner for photoacoustic tomography has a small size of about 1 cm so as to be used for an endoscope, a microscope or a laparoscope for surgery and is capable of performing two-dimensional plane scanning at a high scanning speed of several hundreds of Hz.

A housing and driving member of such a scanner is manufactured by using a PDMS (polydimethyl siloxane) mold as a biomedical polymer material. The PDMS enables a structure having a size of several micrometers to several centimeters to be easily manufactured according to a shape of the mold. In addition, the PDMS enables various types of materials to be fixed by changing the shape of the mold. Namely, if a driving member of the scanner is formed in a flat shape, the PDMS is useful for fixing a mirror reflecting an ultrasonic wave and light, and if a hole is formed at the center of the driving member of the scanner, the PDMS is useful for fixing a micro-sized ultrasonic transducer, optical fiber, or the like. In addition, since the PDMS is flexible in terms of mechanical properties, a mechanical structure made of the PDMS such as a beam spring of a scanner is allowed to have a low spring constant. Therefore, while the size of the scanner is maintained small, a large tilting angle can be obtained by a small electromagnetic force. In addition, due to the flexible property, the PDMS has an additional advantage of being robust against external impacts.

In the above-described scanner, total four permanent magnets are attached to the driving members in a manner that two permanent magnets are attached in each axis direction, and four electromagnets are disposed under the driving members. The driving members perform tilting movement in two-axis directions by applying sine wave voltages at about resonance frequency to the four electromagnets. Herein, in the case where the resonance frequency and the frequencies of the applied voltages are coincident with each other, the angle of the tilting of the driving members of the scanner can be maximized.

In this manner, in the invention, the driving members of the scanner perform tilting movement in the two-axis directions, and a field of view is determined according to the tilting movement, so that, in comparison with a linear-stage-based scanning system, it is possible to acquire a wide range of a three-dimensional photoacoustic tomography image while a small size of the scanner is maintained.

### First Embodiment

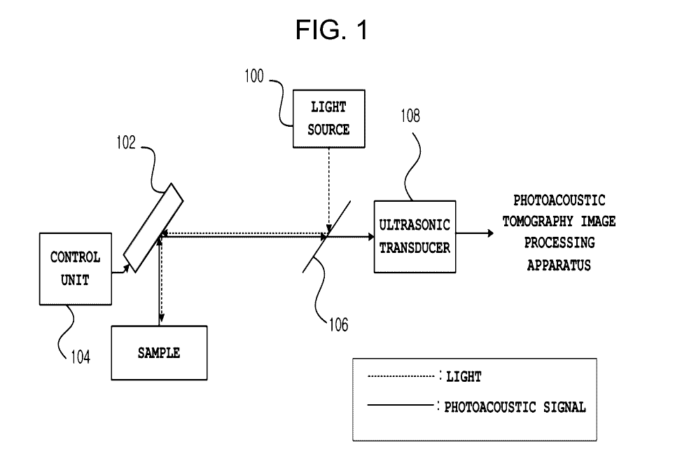

An overall configuration of a photoacoustic tomography apparatus according to the first embodiment of the invention will be described with reference to FIG. 1.

The photoacoustic tomography apparatus is configured to include a light source 100, a scanner 102, a control unit 104, a dichroic mirror 106, and an ultrasonic transducer 108.

The light source 100 generates pulse laser as light for light for photoacoustic tomography and irradiates the dichroic mirror 106 with the pulse laser.

The dichroic mirror 106 is a member of reflecting only a specific wavelength of light. If the pulse laser is incident, the dichroic mirror reflects the pulse laser to a predetermined direction to transfer the pulse laser to a mirror surface of the driving member of the scanner 102. In addition, the dichroic mirror passes the photoacoustic signal transferred from the scanner 102 and transfers the photoacoustic signal to the ultrasonic transducer 108. Herein, instead of the dichroic mirror 106, a beam splitter or the like may be used.

The scanner 102 allows driving members changing an angle of a reflecting surface of the mirror to tilt in two-axis directions under the control of the control unit 104 for capturing a three-dimensional image to change a reflection path of the pulse laser or the photoacoustic signal. Namely, the scanner 102 reflects the pulse laser from the dichroic mirror 106 to transfer the pulse laser to a sample and reflects the photoacoustic signal generated from the sample absorbing the pulse laser to transfer the photoacoustic signal to the dichroic mirror 106, so that the reflection path for capturing the three-dimensional image is changed.

The control unit 104 controls the tilting of the driving member changing the angle of the reflecting surface of the mirror provided to the scanner 102 to allow the surface of the mirror to be tilted in the two-axis directions.

The ultrasonic transducer 108 acquires a photoacoustic signal generated at the time of the sample absorbing pulse laser, the absorbed energy being changed into heat, and the heat being changed into pressure, and transmits the photoacoustic signal to a photoacoustic tomography image processing apparatus (not shown) . The photoacoustic tomography image processing apparatus processes the photoacoustic signal to generate photoacoustic tomography image information. The process of generating the photoacoustic tomography image information is well-known, and thus, the detailed description is omitted.

<Configuration of Scanner 102>

A configuration and operations of the scanner 102 applied to the photoacoustic tomography apparatus configured as described above according to the first embodiment of the invention will be described more in detail.

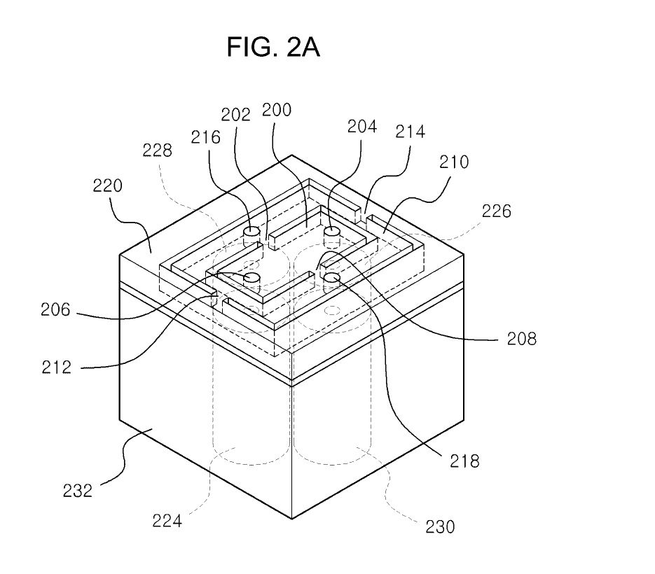

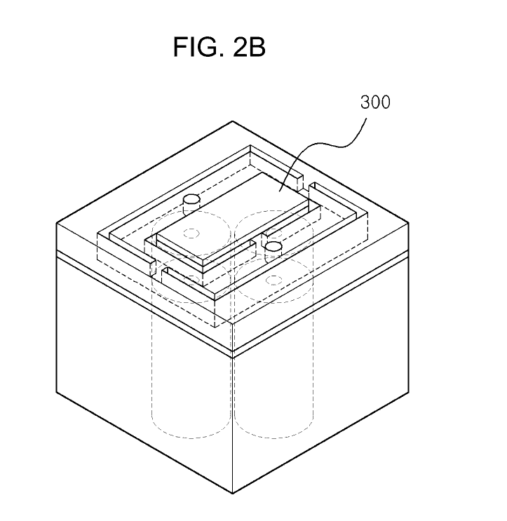

FIGS. 2A and 2B illustrate a structure of the scanner 102.

An outer housing 232 of the scanner 102 is made of PDMS. A flat-shaped mirror 300 is disposed on the top surface of the outer housing 232, and first and second mirror driving members 200 and 210 for performing tilting movement in the X or Y axis are provided. The first mirror driving member 200 has a rectangular flat shape, and the second mirror driving member 210 has a rectangular frame shape where the first mirror driving member 200 is accommodated inside the rectangular frame of the second mirror driving member 210. A top surface member 220 of the outer housing 232 is arranged outside the second mirror driving member 210. The first mirror driving member 200 and the second mirror driving member 210 are connected to each other through first and second connection members 202 and 208. The second mirror driving member 210 and the top surface member 220 are connected to each other through third and fourth connection members 212 and 214. In the scanner 102, since the top surface member 220 of the outer housing 232, the first and second mirror driving members 200 and 210, and the first to fourth connection members 202, 208, 212, and 214 are made of PDMS having flexibility and elasticity, even in the connection state, tilting movement can be performed.

The flat-shaped mirror 300 is attached to the top surface of the first mirror driving member 200.

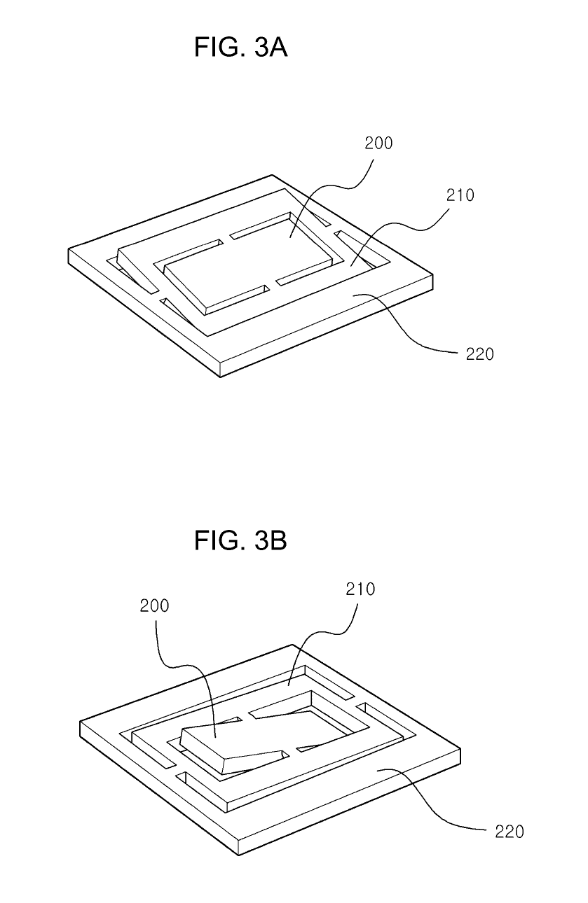

The first mirror driving member 200 is provided in order to perform Y-directional tilting movement as illustrated in FIG. 3B, and first and second permanent magnets 204 and 206 are attached on the bottom surfaces of two ends of the first mirror driving member in the Y direction. First and second electromagnets 224 and 226 are disposed under the respective first and second permanent magnets 204 and 206. As the first and second electromagnets 224 and 226 are supplied with driving power, an attractive force or a repulsive force is generated between the first and second permanent magnets 204 and 206 and the respective first and second electromagnets 224 and 226, and thus, the first and second permanent magnets 204 and 206 are moved upward and downward. Therefore, the first mirror driving member 200 attached with the first and second permanent magnets 204 and 206 performs the Y-directional tilting movement.

The second mirror driving member 210 is connected to the first mirror driving member while accommodating the first mirror driving member 200 inside thereof.

The second mirror driving member 210 is provided in order to perform X-directional tilting movement as illustrated in FIG. 3A, and third and fourth permanent magnets 216 and 218 are attached on the bottom surfaces of two ends of the second mirror driving member in the X direction. Third and fourth electromagnets 228 and 230 are disposed under the respective third and fourth permanent magnets 216 and 218. As the third and fourth electromagnets 228 and 230 are supplied with driving power, an attractive force or a repulsive force is generated between the third and fourth permanent magnets 216 and 218 and the respective third and fourth electromagnets 228 and 230, and thus, the third and fourth permanent magnets 228 and 230 are moved upward and downward. Therefore, the second mirror driving member 210 attached with the third and fourth permanent magnets 216 and 218 performs the X-directional tilting movement.

Each of the first to fourth electromagnets 224, 226, 228, and 230 is driven according to a driving signal supplied by the control unit 104 to exert an attractive force or a repulsive force to the respective facing first to fourth permanent magnets 204, 206, 216, and 218.

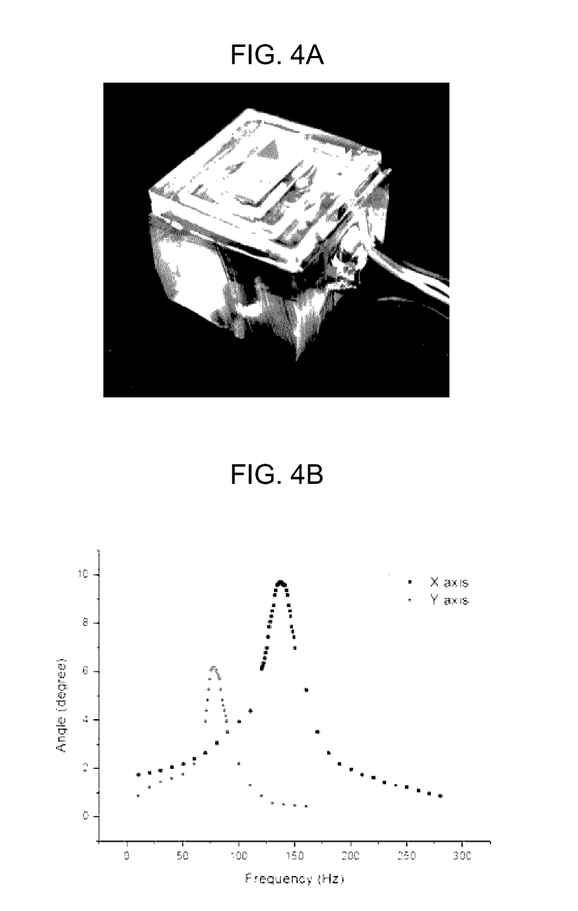

An example of an actual product of the scanner 102 having the above-described configuration is illustrated in FIG. 4A. FIG. 4B illustrates an angle of orientation changing according to a frequency of the driving signal supplied to the first to fourth electromagnets 224, 226, 228, and 230. According to results of an experiment with the actual product of the scanner 102, it was found that resonance frequencies in the directions were 70 Hz and 140 Hz and maximum scanning ranges in the directions were 6.5 degrees and 10 degrees.

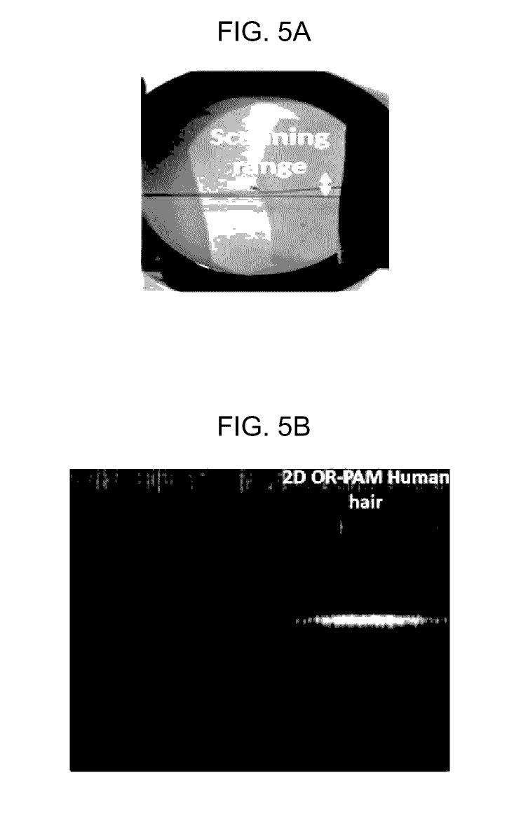

FIG. 5A illustrates a string of hair of a person, and FIG. 5B illustrates a result of scanning of the string of hair in water by using the photoacoustic tomography apparatus according to the embodiment of the invention.

<Process of Driving Scanner>

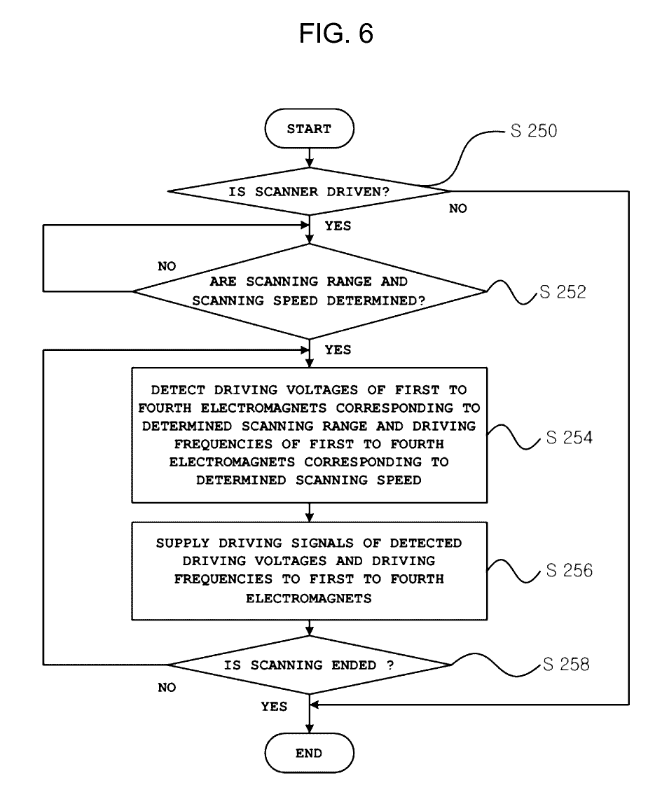

A process of driving the scanner 102 according to the first embodiment of the invention will be described with reference to FIG. 6.

When scanner driving is requested from an outside (step 250), the control unit 104 determines a scanning range and a scanning speed (step 252). Herein, a scanner driving command, information of scanning ranges, and information of scanning speeds may be input through a user interface (not shown).

If the control unit 104 determines the scanning ranges and the scanning speeds, the control unit detects driving voltages of the first to fourth electromagnets 224, 226, 228, and 230 corresponding to the scanning range and detects driving frequencies of the first to fourth electromagnets 224, 226, 228, and 230 corresponding to the scanning speed (step 254). Herein, information of the driving voltages of the first to fourth electromagnets 224, 226, 228, and 230 corresponding to the scanning ranges and information of the driving frequencies of the first to fourth electromagnets 224, 226, 228, and 230 corresponding to the scanning speeds may be acquired through experiments or the like in advance and stored in a memory to be read.

If the control unit detects the driving voltages and frequencies of first to fourth electromagnets 224, 226, 228, and 230, the control unit 104 supplies driving signals of the driving voltages and frequencies to the respective first to fourth electromagnets 224, 226, 228, and 230 (step 256). Therefore, the first and second mirror driving members 200 and 210 of the scanner 102 performs two-axis tilting movement in accordance with the scanning ranges and the scanning speeds. Accordingly, a reflecting angle of the mirror attached to the first mirror driving member 200 is changed, and thus, the scanning is performed in accordance with the scanning range and the scanning speed.

### Second Embodiment

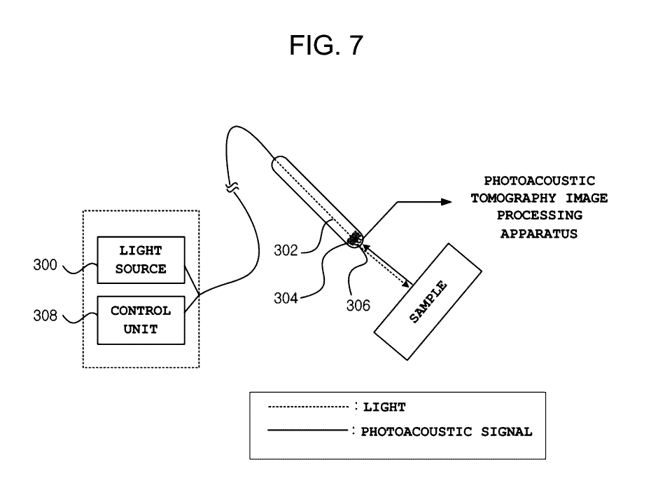

An overall configuration of a photoacoustic tomography apparatus manufactured in a form of an endoscope according to the second embodiment of the invention will be described with reference to FIG. 7.

The photoacoustic tomography apparatus is configured to include a light source 300, an optical fiber 302, a scanner 304, a control unit 308, and an ultrasonic transducer 306.

The light source 300 generates pulse laser as light for photoacoustic tomography, and the pulse laser is incident on the optical fiber 302.

A light incident surface of the optical fiber 302 is connected to a pulse laser light emitting surface of the light source 300, and a light emitting surface of the optical fiber 302 is positioned at a hole formed at the center of a photoacoustic signal receiving surface of the ultrasonic transducer 306 attached at the center of the top surface of the scanner 304. Therefore, the optical fiber 302 receives the pulse laser from the light source 300 and emits the pulse laser through the light emitting surface positioned at the center of the photoacoustic signal receiving surface of the ultrasonic transducer 306.

In order to acquire a three-dimensional image, the scanner 304 allows the photoacoustic signal receiving surface of the ultrasonic transducer 306 and the light emitting surface of the optical fiber 302 to perform tilting movement in two axis directions under the control of the control unit 308, and thus, the emitting angle of the pulse laser or the receiving angle of the photoacoustic signal is changed. Namely, the scanner 304 transmits the pulse laser emitted through the light emitting surface of the optical fiber 302 to a sample, and the photoacoustic signal generated from the sample absorbing the pulse laser is incident on the ultrasonic transducer 306. At this time, the emitting angle and the receiving angle are allowed to be changed simultaneously, so that the three-dimensional image can be acquired.

The control unit 308 controls the tilting movement of the driving members provided to the scanner 304, so that the photoacoustic signal receiving surface of the ultrasonic transducer 306 or the light emitting surface of the optical fiber 302 is allowed to perform the tilting movement in two axis directions.

The ultrasonic transducer 306 is attached on the driving member of the scanner 304, the angle of the photoacoustic signal receiving surface is changed according to the two-axis tilting movement of the driving member. When the sample absorbs the pulse laser, the absorbed energy of the pulse laser is converted into heat, and the heat is converted to pressure.

When the heat is converted to the pressure, the photoacoustic signal is generated, and the ultrasonic transducer 306 acquires the photoacoustic signal and transmits the photoacoustic signal to a photoacoustic tomography image processing apparatus (not shown). The photoacoustic tomography image processing apparatus processes the photoacoustic signal to generate photoacoustic tomography image information.

A hole is formed at the center of the photoacoustic signal receiving surface of the ultrasonic transducer 306, and the optical fiber 302 is inserted into the hole to be coupled. Therefore, the emitting angle of the pulse laser can be coincident with the receiving angle of the photoacoustic signal.

Particularly, the scanner 304 and the ultrasonic transducer 306 are accommodated into the end portion of a housing of the endoscope.

<Configuration of Scanner 304>The configuration and operations of the scanner 4 applied to the photoacoustic tomography apparatus having the above-described configuration according to the second embodiment of the invention will be described more in detail.

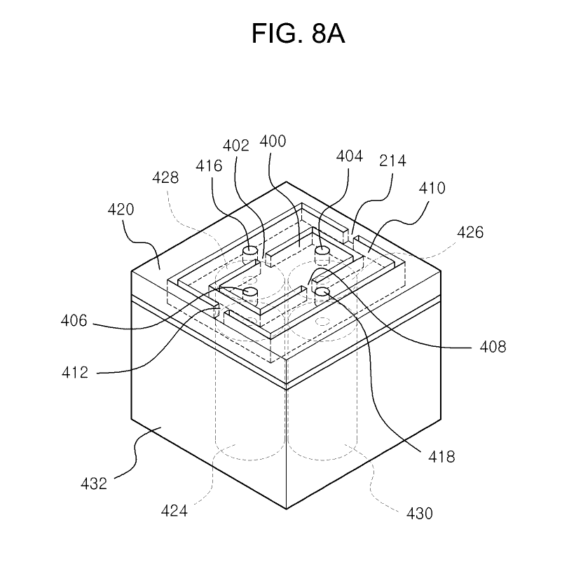

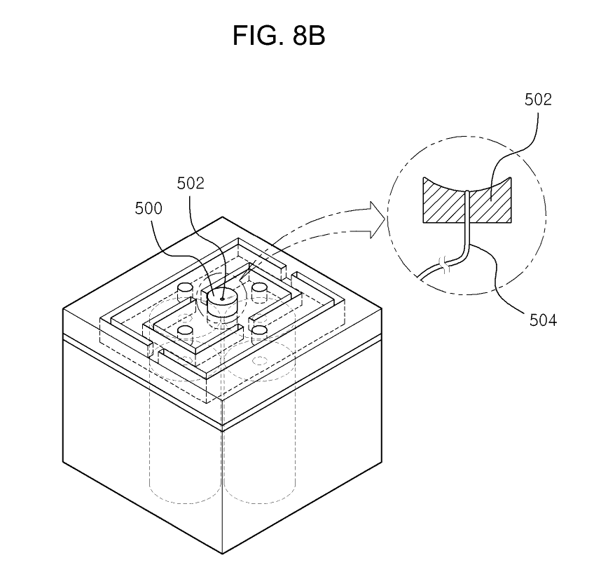

FIGS. 8A and 8B illustrate a structure of the scanner 304.

An outer housing 432 of the scanner 304 is made of PDMS. The ultrasonic transducer 306 and the optical fiber are disposed on the top surface of the outer housing 432, and first and second driving members 400 and 410 for performing tilting movement in the X or Y axis are provided. The first driving member 400 has a rectangular frame shape, and the second driving member 410 has a rectangular frame shape where the first driving member 400 is accommodated inside the frame of the second driving member 410.

A top surface member 420 of the outer housing 432 is arranged outside the second driving member 410. The first driving member 400 and the second driving member 410 are connected to each other through first and second connection members 402 and 408. The second driving member 410 and the top surface member 420 are connected to each other through third and fourth connection members 412 and 414 . In the scanner 304, since the top surface member 420 of the outer housing 432, the first and second driving members 400 and 410, and the first to fourth connection members 402, 408, 412, and 414 are made of having flexibility and elasticity, even in the connection state, tilting movement can be performed.

The ultrasonic transducer 306 is attached on the top surface of the first driving member 400, and the optical fiber 302 is inserted into the center of the photoacoustic signal receiving surface of the ultrasonic transducer 306.

The first driving member 400 is provided in order to perform Y-directional tilting movement, and first and second permanent magnets 404 and 406 are attached on the bottom surfaces of two ends of the first driving member in the Y direction. First and second electromagnets 424 and 426 are disposed under the respective first and second permanent magnets 404 and 406. As the first and second electromagnets 424 and 426 are supplied with driving power, an attractive force or a repulsive force is generated between the first and second permanent magnets 404 and 406 and the respective first and second electromagnets 424 and 426, and thus, the first and second permanent magnets 404 and 406 are moved upward and downward. Therefore, the first driving member 400 performs the Y-directional tilting movement.

The second driving member 410 is connected to the first driving member 400 while accommodating the first driving member 400 inside thereof.

The second driving member 410 is provided in order to perform X-directional tilting movement, and third and fourth permanent magnets 416 and 418 are attached on the bottom surfaces of two ends of the second driving member in the X direction. Third and fourth electromagnets 428 and 430 are disposed under the respective third and fourth permanent magnets 416 and 418. As the third and fourth electromagnets 428 and 430 are supplied with driving power, an attractive force or a repulsive force is generated between the third and fourth permanent magnets 428 and 430 and the respective third and fourth electromagnets 428 and 430, and thus, the third and fourth permanent magnets 428 and 430 are moved upward and downward. Therefore, the second driving member 410 performs the X-directional tilting movement.

Each of the first to fourth electromagnets 424, 426, 428, and 430 is driven according to a driving signal supplied by the control unit 308 to exert an attractive force or a repulsive force to the respective facing first to fourth permanent magnets 404, 406, 416, and 418.

When scanner driving is requested from an outside, the control unit 308 controlling the scanner 304 according to the second embodiment of the invention determines a scanning range and a scanning speed. If the control unit determines the scanning range and the scanning speed, the control unit detects driving voltages of the first to fourth electromagnets 424, 426, 428, and 430 corresponding to the determined scanning range and detects driving frequencies of the first to fourth electromagnets 424, 426, 428, and 430 corresponding to the determined scanning speed. If the control unit detects the driving voltages and driving frequencies of the first to fourth electromagnets 424, 426, 428, and 430, the control unit 104 supplies driving signals of the driving voltages and the driving frequencies to the first to fourth electromagnets 424, 426, 428, and 430. Therefore, the scanner 304 is moved in two axes in accordance with the scanning range and the scanning speed.

In addition, in the above-described second embodiment of the invention, although only the example where the optical fiber 302 is inserted into the center of the photoacoustic signal receiving surface of the ultrasonic transducer 306 and the ultrasonic transducer 306 and the optical fiber 302 are integrated is exemplified, it is obvious to the skilled in the art that the scanner may be configured to include only the ultrasonic transducer or to include only the optical fiber.

In addition, in the above-described embodiments of the invention, although only the example where the permanent magnets are attached to the driving members and the electromagnets are disposed under the driving members, it is obvious to the skilled in the art that the permanent magnets and the electromagnets can be exchanged in terms of position.

In addition, in the above-described embodiments of the invention, although only the example where the attractive force or a repulsive force between only the pair of the electromagnet and the permanent magnet are employed for the driving force for the tilting movement is exemplified, it is obvious to the skilled in the art that a pair of electromagnets may be employed.

In addition, in the above-described embodiments of the invention, although only the example where the attractive force or a repulsive force between only the pair of the electromagnet and the permanent magnet are employed for the driving force for the tilting movement is exemplified, it is obvious to the skilled in the art that a pair of an electromagnet and a magnetic material such as iron may be employed, and in this case, the driving force is an attractive force between the electromagnet and the magnetic material.

In addition, in the above-described embodiments of the invention, although only the example where the attractive force or a repulsive force between only the pair of the electromagnet and the permanent magnet are employed for the driving force for the tilting movement is exemplified, it is obvious to the skilled in the art that a pair of metal electrodes may be employed, and in this case, the driving force is an attractive force between the metal electrodes in the pair.

In addition, in the above-described embodiments of the invention, although only the example where the driving members of the scanner performs tilting movement in two axes for three-dimensional scanning is exemplified, it is obvious to the skilled in the art that only one of the driving members of the scanner may selectively perform tilting movement for two-dimensional scanning.

In addition, the scanner according to the invention can be manufactured in a small size so as to be used for an endoscope, a microscope or a laparoscope for surgery. The scanner according to the invention enables an endoscope to image a sentinel lymph node located under a stomach wall from which spread of a stomach cancer can be determined in diagnosis of the stomach cancer.

While the present invention has been particularly shown and described with reference to exemplary embodiments thereof, it will be understood by those skilled in the art that various changes in form and details maybe made therein without departing from the spirit and scope of the present invention as defined by the appended claims.

1. A scanner for photoacoustic tomography comprising:
a mirror which reflects light and a photoacoustic signal for the photoacoustic tomography;
a first driving member which is attached with the mirror;
a second driving member which is connected to the first driving member;
first driving force supply units which are disposed under two ends of the first driving member in a first direction to exert a driving force for allowing the first driving member to perform tilting movement in the first direction; and
second driving force supply units which are disposed under two ends of the second driving member in a second direction to exert a driving force for allowing the second driving member to perform tilting movement in the second direction.

2. (canceled)

3. (canceled)

4. (canceled)

5. The scanner according to claim 1,
wherein the first and second driving force supply units are configured with a pair of a permanent magnet and an electromagnet,
wherein the permanent magnet and the electromagnet in the pair face each other,
wherein one of the permanent magnet and the electromagnet is positioned to be attached to the first and second driving member, and the other is positioned to be separated from the first and second driving member,
wherein the driving force is generated as a driving signal is applied to the electromagnet in the pair of the permanent magnet and the electromagnet, and
wherein the driving force is an attractive force and a repulsive force between the permanent magnet and the electromagnet.

6. The scanner according to claim 1,
wherein the first and second driving force supply units are configured with a pair of an electromagnet and a magnetic material,
wherein the electromagnet and the magnetic material in the pair face each other,
wherein one of the electromagnet and the magnetic material is positioned to be attached to the first and second driving member, and the other is positioned to be separated from the first and second driving member,
wherein the driving force is generated as a driving signal is applied to the electromagnet in the pair of the electromagnet and the magnetic material, and
wherein the driving force is an attractive force between the electromagnet and the magnetic material.

7. The scanner according to claim 1,
wherein the first and second driving force supply units are configured with a pair of metal electrodes,
wherein the metal electrodes in the pair face each other,
wherein one of the metal electrodes is attached to the first and second driving member, and the other is positioned to be separated from the first and second driving member,
wherein the driving force is generated by applying a driving signal to the pair of the metal electrodes, and
wherein the driving force is an attractive force between the metal electrodes in the pair.

8. The scanner according to claim 1, further comprising an outer housing constituting an outer surface of the scanner,
wherein the outer housing and the first and second driving member are formed with a biomedical polymer material.

9. The scanner according to claim 8, wherein the biomedical polymer material is PDMS (polydimethyl siloxane).

10. A photoacoustic tomography apparatus for an endoscope comprising the scanner according to claim 1.

11. A photoacoustic tomography apparatus for a microscope comprising the scanner according to claim 1.

12. A photoacoustic tomography apparatus for a laparoscope for surgery, comprising the scanner according to claim 1.
